(19) United States
(12) Patent Application Publication
(10) Pub. No.: US 2024/0398315 A1
(43) Pub. Date: Dec. 5, 2024

(54) APPARATUS FOR PREDICTING ATRIAL FIBRILLATION BASED ON ARTIFICIAL INTELLIGENCE, AND OPERATING METHOD THEREOF

(71) Applicants: SAMSUNG LIFE PUBLIC WELFARE FOUNDATION, Seoul (KR); Wellysis Corp., Seoul (KR)

(72) Inventors: Kyoung-Min PARK, Seoul (KR); Sunghee SONG, Incheon (KR); Hongryul KIM, Seoul (KR); Jongwoo KIM, Seoul (KR); Ji-Hoon CHOI, Seoul (KR)

(21) Appl. No.: 18/636,402

(22) Filed: Apr. 16, 2024

(30) Foreign Application Priority Data
May 30, 2023 (KR) 10-2023-0069397

(51) Int. Cl. A61B 5/361; A61B 5/00; A61B 5/339; A61B 5/353; A61B 5/355; A61B 5/358; A61B 5/36; G16H 10/60; G16H 20/10; G16H 50/70

(52) U.S. Cl. CPC A61B 5/361; A61B 5/339; A61B 5/353; A61B 5/7267; A61B 5/7275; G16H 10/60; G16H 20/10; G16H 50/70; A61B 5/355; A61B 5/358; A61B 5/36

(57) ABSTRACT

An apparatus for predicting atrial fibrillation (AF) operated by at least one processor includes an electrocardiogram (ECG) preprocessor configured to acquire individual ECG pairs measured at a certain period of time and generate a difference between the ECG pairs as input data for an artificial intelligence model, an artificial intelligence model configured to be trained to predict an onset-AF possibility from an ECG difference and output a probability of onset-AF predicted from the input data, and a prediction information provider configured to provide an AF prediction including the probability of onset-AF to a designated device.

### CROSS-REFERENCE TO RELATED APPLICATION

This application claims priority to and the benefit of Korean Patent Application No. 10-2023-0069397 filed in the Korean Intellectual Property Office on May 30, 2023, the entire contents of which are incorporated herein by reference.

### BACKGROUND

### (a) Field

The present disclosure relates to an atrial fibrillation (AF) prediction.

### (b) Description of the Related Art

Atrial fibrillation (AF) is the most prevalent arrhythmia, and the number of affected patients with AF is rapidly increasing, resulting in substantial healthcare costs.

It is known that the patients with AF have a five-fold higher risk of stroke compared to normal people, and 30% of patients with AF experience stroke at least once during their lives. According to neurology reports, approximately 30-40% of patients hospitalized for acute stroke were related to AF. It is known that about 50% of the hospitalized patients are often discovered through electrocardiogram (ECG) examination or long-term ECG monitoring after hospitalization rather than before hospitalization.

The key to preventing stroke in patients with AF is anticoagulation treatment through early diagnose. However, the early diagnosis is difficult because the AF often appears spasmodically and briefly in the early stages.

### SUMMARY

The present disclosure attempts to provide an apparatus for predicting atrial fibrillation (AF) and an operating method thereof.

The present disclosure relates to an artificial intelligence model that predicts a new onset-AF of a patient from changes in electrocardiograms (ECGs).

According to an embodiment, an apparatus for predicting atrial fibrillation (AF) operated by at least one processor includes an electrocardiogram (ECG) preprocessor configured to acquire individual ECG pairs measured at a certain period of time and generate a difference between the ECG pairs as input data for an artificial intelligence model, an artificial intelligence model configured to be trained to predict a possibility of onset-AF from an ECG difference and output a probability of onset-AF predicted from the input data, and a prediction information provider configured to provide an AF prediction including the probability of onset-AF to a designated device.

The ECG preprocessor may be configured to extract ECG features from ECG signals of each ECG included in the ECG pair, and generate a feature difference between the two ECGs as the input data.

The ECG features may include P-QRS-T waveform features.

The ECG features may further include at least one of ECG beat similarity, fibrillatory wave (F-wave) energy, and P-wave features.

The input data may further include at least one of an individual gender and age.

The artificial intelligence model may be trained to learn a relationship between an input and a label tagged in the input using training data to output a value between 0 and 1 predicted for the input data. The training data may be generated using ECGs of patients whose first ECG and second ECG measured at a first visit and a second visit are normal, and whose third ECG measured at a third visit is normal or has AF, the input may be a difference between the first ECG and the second ECG, and the label may be given according to the third ECG of the patient.

The AF prediction may further include a performance indicator for the probability of onset-AF.

The prediction information provider may provide decision-making assistance information related to the AF prediction.

According to another embodiment, an operating method of an apparatus for predicting atrial fibrillation (AF) operated by at least one processor includes acquiring individual electrocardiogram (ECG) pair measured at a certain period of time, and predicting a probability of onset-AF for an ECG difference between the ECG pairs using an artificial intelligence model trained to predict a possibility of onset-AF from the ECG difference.

The artificial intelligence model may be trained to learn a relationship between an input and a label tagged in the input using training data to output a value between 0 and 1 predicted for input data. The training data may be generated using ECGs of patients whose first ECG and second ECG measured at a first visit and a second visit are normal, and whose third ECG measured at a third visit is normal or has the AF, the input may be a difference between the first ECG and the second ECG, and the label may be given according to the third ECG of the patient.

The operating method may further include extracting ECG features from ECG signals of each ECG included in the ECG pair, and generating a feature difference between the two ECGs as input data of the artificial intelligence model.

The ECG features may include P-QRS-T waveform features.

The ECG features may further include at least one of ECG beat similarity, fibrillatory wave (F-wave) energy, and P-wave features.

The input data may further include at least one of an individual gender and age.

The operating method may further include predicting the AF including the probability of onset-AF and providing decision-making assistance information related to the AF prediction to a designated device.

The AF prediction may further include a performance indicator for a probability of onset-AF.

According to still another embodiment, an operating method of an apparatus for predicting atrial fibrillation (AF) operated by at least one processor includes identifying whether a patient measuring a current ECG is a returning patient having a past ECG measured in the past or a first-time patient, for the returning patient, obtaining a probability of onset-AF predicted for a past and current ECG difference of the patient using a serial ECG model trained to predict the possibility of onset-AF from the ECG difference, for the first-time patient, obtaining a probability of onset-AF predicted for the current ECG of the patient using a single ECG model trained to predict the possibility of onset-AF from the single ECG, and providing an AF prediction including the probability of onset-AF of the patient to a designated device.

The serial ECG model may be trained to learn a relationship between an input and a label tagged in the input using training data to output a value between 0 and 1 predicted for the ECG difference of the patient. The training data may be generated using ECGs of patients whose first ECG and second ECG measured at a first visit and a second visit are normal, and whose third ECG measured at a third visit is normal or has the AF, the input may be a difference between the first ECG and the second ECG, and the label may be given according to the third ECG of the patient.

The single ECG model may be trained to learn a relationship between an input and a label tagged in the input using training data to output a value between 0 and 1 predicted for the input data. The training data may be generated using ECGs of patients whose first ECG measured at a first visit is normal and whose second ECG measured at a second visit is normal or has the AF, the input may be features of the first ECG, and the label may be given according to the second ECG of the patient.

The operating method may further include providing decision-making assistance information related to the AF prediction to the designated device.

According to the present disclosure, it is possible to accurately select patients being new onset-AF in the future through an evolved artificial intelligence model considering individual characteristics.

According to the present disclosure, it is possible to support medical personnel's decision-making such as an active ECG monitoring test or preemptive anticoagulation treatment for patients with a high probability of onset-AF, and increase the AF diagnosis rate.

According to the present disclosure, it is possible to recommend the appropriate follow up clinical procedures depending on the possibility of onset-AF and determine the optimal follow up period (e.g. 3 months) considering the accuracy of AF prediction.

### BRIEF DESCRIPTION OF THE DRAWINGS

FIG. 1 is a diagram illustrating an apparatus for predicting atrial fibrillation (AF) based on artificial intelligence according to an embodiment.

FIG. 2 is an example of an electrocardiogram (ECG) signal.

FIG. 3 is an example of P-QRS-T analysis.

FIG. 4 is a diagram illustrating a method of calculating ECG beat similarity according to an embodiment.

FIG. 5 is a diagram describing training of an artificial intelligence model according to an embodiment.

FIG. 6 is a diagram illustrating a method of generating training data for a serial ECG model according to an embodiment.

FIG. 7 is a diagram describing a method of generating training data for a single ECG model according to an embodiment.

FIG. 8 is a flowchart illustrating an operating method of an apparatus for predicting AF according to an embodiment.

FIG. 9 is a flowchart illustrating an operating method of an apparatus for predicting AF according to another embodiment.

FIG. 10 is a hardware configuration diagram of the apparatus for predicting AF according to an embodiment.

### DETAILED DESCRIPTION OF THE EMBODIMENTS

Hereinafter, exemplary embodiments of the present disclosure will be described in detail with reference to the accompanying drawings so that those skilled in the art to which the present disclosure pertains may easily practice the present disclosure. However, the present disclosure may be modified in various different forms, and is not limited to embodiments provided in the present specification. In addition, components unrelated to a description will be omitted in the accompanying drawings in order to clearly describe the present disclosure, and similar reference numerals will be used to denote similar components throughout the present specification.

Throughout the specification, unless explicitly described to the contrary, the word “comprise” and variations such as “comprises” or “comprising”, will be understood to imply the inclusion of stated elements but not the exclusion of any other elements. In addition, the terms “-er”, “-or”, and “module” described in the specification mean units for processing at least one function and operation, and can be implemented by hardware components or software components, and combinations thereof.

An electrocardiogram (ECG) is signals obtained through electrodes attached to a body, and an ECG test is a heart test mainly used to detect arrhythmia diseases. A standard 12-lead ECG is obtained through electrodes attached to wrists, ankles, and chest on each side, and includes standard limb leads I, II, and III, augmented unipolar limb leads aVR, aVL, aVF, and chest leads V1, V2, V3, V4, V5, and V6.

With the development of artificial intelligence technology, artificial intelligence technology is being used for various medical predictions, and researches are also being conducted to predict atrial fibrillation (AF) from 12-lead ECGs. Most related arts predict AF using one normal Sinus rhythm (NSR) ECG obtained from one individual. Predicting the AF through the ECG is based on the theoretical background that the NSR ECG of people for whom the AF will develop in the future will be different from those for whom the AF will develop. However, since the shape, size, rotation, relative position to a chest, and component of a body composition of the heart are all different for each individual, even NSR waveforms obtained from healthy adults without cardiovascular disease actually show significant differences from individual to individual. Therefore, it is necessary to predict the AF, which appears as subtle changes that are impossible to distinguish with the human eye. However, since the artificial intelligence model is trained with the NSR waveforms that can be distinguished with the human eye, there are limits to increasing prediction performance beyond a certain level.

Unlike the related art, since the ECG waveform varies from person to person, cardiac remodeling precedes the AF, and these subtle changes cause the changes in ECGs that can only be distinguished at the artificial intelligence level, the present disclosure presents an artificial intelligence model that predicts the future occurrence of AF from these ECG changes.

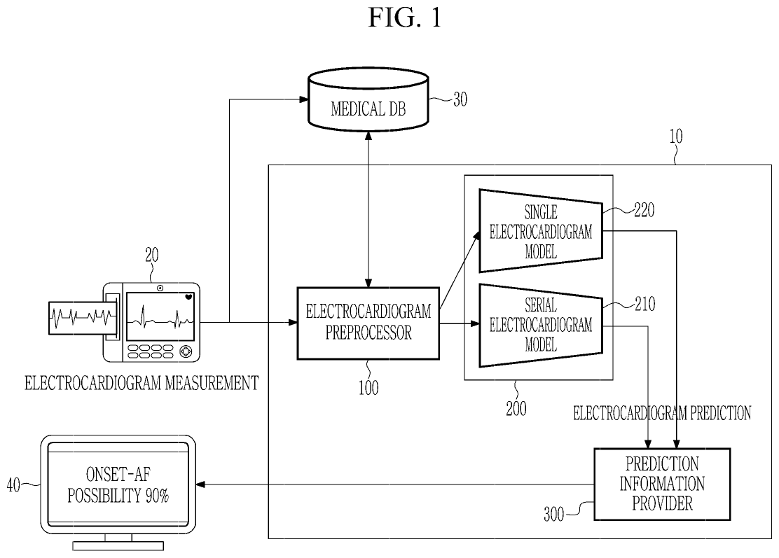

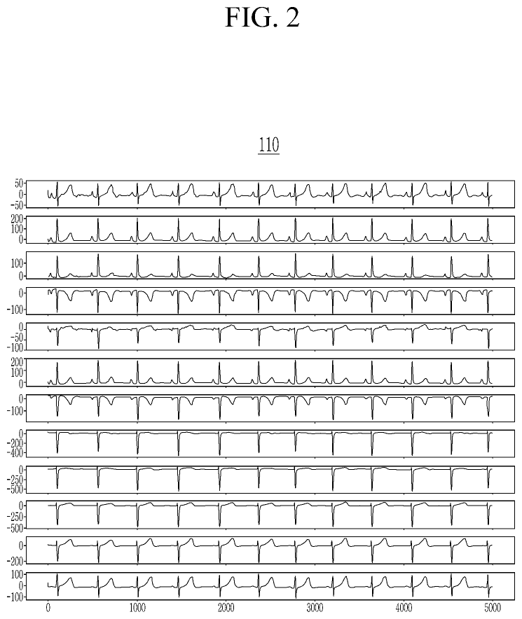

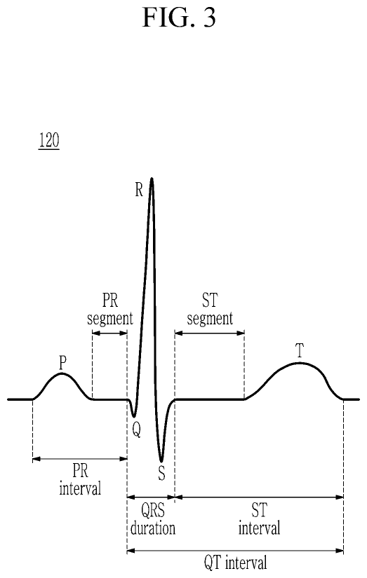

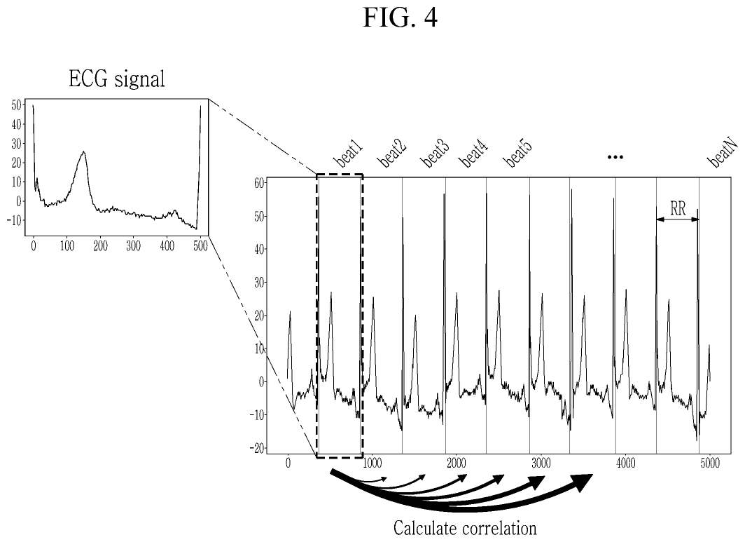

FIG. 1 is a diagram illustrating an apparatus for predicting AF based on artificial intelligence according to an embodiment. FIG. 2 is an example of an electrocardiogram signal. FIG. 3 is an example of P-QRS-T analysis. FIG. 4 is a diagram illustrating a method of calculating ECG beat similarity according to an embodiment.

Referring to FIG. 1, an apparatus 10 for predicting AF operated by at least one processor includes an artificial intelligence (AI) model 200 trained to predict the onset-AF (AF occurrence) from an ECG. The apparatus 10 for predicting AF outputs an AF prediction including onset-AF possibility of a patient. The possibility of onset-AF in the future may be predicted from the past and present changes in ECGs. The apparatus 10 for predicting AF may predict new-onset AF which will occur in the future, although the ECG is currently normal, through the artificial intelligence model 200.

The apparatus 10 for predicting AF may provide the possibility of onset-AF in a patient on an interface screen of a terminal 40 and may additionally provide decision-making assistance information on the interface screen of the terminal 40. The apparatus 10 for predicting AF may be implemented as a computing device equipped with a program for performing the present disclosure and hardware for executing the program. The computing device may operate as the apparatus 10 for predicting AF by installing and executing the program for carrying out the present disclosure.

The apparatus 10 for predicting AF may be built to be linked with at least one ECG measurement device 20, a medical database (DB) 30 including patient information and an ECG database, and at least one terminal 40 through a wired or wireless network. In addition, the apparatus 10 for predicting AF may be constructed in various forms. For example, the apparatus 10 for predicting AF may be constructed integrally with the ECG measurement device 20, or may be constructed individually for each local site where the ECG measurement device 20 is located.

The apparatus 10 for predicting AF may include an ECG preprocessor 100 that generates input data of the artificial intelligence model 200 from the ECG signals, and the artificial intelligence model 200 that predicts the onset-AF from the input data. The apparatus 10 for predicting AF may further include a prediction information provider 300 that provides the terminal 40 with the decision-making assistance information related to the onset-AF possibility predicted from the ECG of the patient and the AF prediction.

The ECG preprocessor 100 may receive the ECG data measured in real time from the ECG measurement device 20 or receive the ECG data stored in the medical database 30.

The ECG preprocessor 100 may extract ECG features from ECG signals of a patient and generate input data suitable for the artificial intelligence model 200 using the ECG features. The input data of the artificial intelligence model 200 may be variously defined by a combination of the ECG features. As the ECG features, P-QRS-T wave features, ECG beat similarity, fibrillatory wave (F-wave) energy, P-wave features, etc., may be used. The operation of the ECG preprocessor 100 will be described in detail below. The ECG preprocessor 100 may generate training data for the artificial intelligence model 200.

The artificial intelligence model 200 outputs the AF prediction including the possibility of onset-AF, and the possibility of onset-AF may be provided together with the time of occurrence. The possibility of onset-AF may be expressed in terms of probability, score, risk (high, medium, low), etc., and may mainly be explained using probability as an example. The time of occurrence of AF may be provided, for example, after 3 months, after 6 months, after 1 year, etc. The AF prediction output from the artificial intelligence model 200 may variously change depending on the training data.

The artificial intelligence model 200 may be implemented as a variety of models that can be trained to output information on the onset-AF from the input. For example, the artificial intelligence model 200 may use both decision tree-based bagging and boosting-series models, and the boosting-series model has relatively higher prediction accuracy than the bagging model. Among the boosting series, a gradient boosting machine (GBM), a light gradient boosting machine (LGBM), which can be used even if some of the input variables are missing, XGBoost, etc., may be used. A boosting-based artificial intelligence model may output information on onset-AF predicted after a certain period of time (e.g., 1 month) based on a boosting decision tree.

The artificial intelligence model 200 may include a serial ECG model 210 trained to predict the onset-AF possibility from the changes in ECGs. Here, the changes in ECGs may be acquired from individual ECG pairs measured at a certain period of time. The artificial intelligence model 200 may further include a single ECG model 220 trained to predict the onset-AF possibility from the current ECG. Since the serial ECG model 210 predicts the AF from subtle changes before the AF occurs, the prediction accuracy may be superior to the single ECG model 220. Therefore, the apparatus 10 for predicting AF may identify whether the patient is a returning patient who has measured the ECG in the past or a first-time patient, and for the returning patient, may obtain the probability of onset-AF using the serial ECG model 210. In addition, for the first-time patient, the apparatus 10 for predicting AF may obtain the probability of onset-AF using the single ECG model 220.

The artificial intelligence model 200 may use normal Sinus rhythm (NSR) ECGs as the training data. The input data and label constituting the training data may be generated differently depending on the model. The NSR ECG may be called a normal ECG.

For example, the serial ECG model 210 is trained using the ECGs of patients whose ECGs were measured at three or more visits. Here, the ECGs measured at the first and second visits may be the NSR, and the ECG measured at the third visit may be the NSR or AF may be used. In this case, an NSR ECG difference between the first and second visits is the input data. When the ECG of the third visit is the NSR, the input data may be tagged with label 0, and when the ECG of the third visit is the AF, the input data may be tagged with label 1. The diagnosis (NSR or AF) on the ECG may be included in the ECG data or indicated in the medical record of the patient.

The single ECG model 220 is trained using the ECGs of patients whose ECGs were measured at twice or more visits. Here, the ECG measured at the first visit may be the NSR, and the ECG measured at the second visit is the NSR or AF may be used. In this case, an NSR ECG feature of the first visit is the input data. When the ECG of the second visit is the NSR, the input data may be tagged with label 0, and when the ECG of the second visit is AF, the input data may be tagged with label 1.

The prediction information provider 300 may provide the AF prediction of the patient output from the artificial intelligence model 200 and the decision-making assistance information related to the AF prediction to the terminal 40. The AF prediction may include the probability of onset-AF, performance indicators for predicted values, etc. The performance indicators may include, for example, sensitivity, specificity, accuracy, a positive predictive value (PPV), and a negative predictive value (NPV). The AF prediction may include the probability of onset-AF for at least one period. The decision-making assistance information is information to assist medical personnel in diagnosis and decision making and may include recommended follow up clinical procedures based on the AF prediction. The follow up clinical procedures may include, for example, an active electrocardiogram monitoring test, follow up termination without further testing, re-performing an electrocardiogram test after a certain period of time, etc. For example, for a patient with a high probability of onset-AF, the apparatus 10 for predicting AF may recommend the active ECG monitoring test that measures the ECG while living with a wearable device and performs monitoring using the measured ECG.

The apparatus 10 for predicting AF may support medical personnel to select patients for whom the AF will develop in the future through the apparatus 10 for predicting AF. In addition, the apparatus 10 for predicting AF may support medical personnel's decision-making such as active electrocardiogram monitoring test or preemptive anticoagulation treatment for patients with a high probability of onset-AF, and increase the AF diagnosis rate. Patients may prepare in advance for the onset-AF, thereby preventing dangerous complications such as stroke caused by the AF.

Next, a method of extracting, by the ECG preprocessor 100, ECG features to generate the input data for the artificial intelligence model 200 will be described in detail.

The ECG preprocessor 100 receives ECG data of a patient. The ECG data is generally raw data including signal information measured through 12-lead ECGs and basic information of the patient, and may be stored in an XML file format. In the case of the normal ECG, information such as Sinus rhythm may be input to a <Statement> tag within a <Diagnosis> tag in XML data, and for the abnormal ECG, a different disease name may be input. The XML data stores measurement values, and the measurement values may be converted into ECG waveforms.

The ECG preprocessor 100 may generate an ECG signal from the ECG data. The ECG preprocessor 100 may connect a label included in the ECG data to the ECG signal. Referring to FIG. 2, an ECG signal 110 may be expressed as a signal waveform measured in 12-leads I, II, III, aVR, aVL, aVF, V1, V2, V3, V4, V5, and V6.

The ECG preprocessor 100 may remove noise included in the ECG signal. Even if the ECG is measured normally, the noise may be included rarely. For example, the ECG signal may include power line interference or baseline wander. The power line interference is mechanical noise that is mixed in all signals and is widely distributed in a 50 to 60 Hz frequency range. The baseline wander appears in the form of a fluctuating waveform, and is largely included in frequencies of 1 Hz or less. Therefore, noise included in the ECG signal may be removed through a 0.5 to 45 Hz band-pass filter. In addition, known noise removal methods may be used.

The ECG preprocessor 100 may remove an outlier included in the ECG signal. The outliers may also exist even in the ECG signal from which the noise has been removed. The ECG preprocessor 100 may calculate a Z-score called a standard-score, for the data points constituting the ECG signal, and consider a data point greater than a threshold (e.g., 10) as the outlier and remove it. The Z-score shows how far the data point is away from mean.

The ECG preprocessor 100 may extract the features (ECG features) of the ECG signal as follows.

The ECG preprocessor 100 may extract the wave features of the ECG signal. The waveform features may be composed of statistics of variables extracted from the P-QRS-T waveform. The statistics may be calculated as at least one of, for example, a minimum value, a maximum value, mean, a standard deviation, and may be calculated by various statistical functions such as median.

First, the ECG signal periodically repeats a unit waveform 120 with a P-wave, a QRS complex, and a T wave, as illustrated in FIG. 3. The P-wave is a waveform that appears when a stimulus delivered to an atrium depolarizes the atrium. The QRS complex, which is composed of a Q wave, an R wave, and an S wave, is a waveform generated by ventricular depolarization. The T wave is a waveform that appears in repolarization of a ventricle. A PR interval is an interval from a start of the P-wave to a start of the QRS, and a PR segment is an interval from an end of the P-wave to the start of QRS. An ST interval is the interval from an end of the S wave to an end of the T wave, and an ST segment is an interval from the end of the S wave to a start of the T wave. A QRS duration is a duration from the Q wave to the S wave. A QT interval is an interval from a start of the Q wave to the end of the T wave.

The ECG preprocessor 100 may first extract the R wave, and extract the P-wave, the QRS complex, and the T wave in order. The ECG preprocessor 100 may measure values of variables (voltage, interval, segment, duration, etc., of each wave) for each unit waveform and use the statistics of each variable as the waveform features. The statistics may include the minimum value, the maximum value, the mean, the standard deviation, etc.

The ECG preprocessor 100 may obtain waveform features as shown in Table 1. The waveform features may include P-peak statistics, Q-peak statistics, R-peak statistics, S-peak statistics, and T-peak statistics, and are statistics for peak amplitude values of each P-QRS-T wave. The waveform features may include PP interval statistics, PR interval statistics, QQ interval statistics, RR interval statistics, SS interval statistics, TT interval statistics, and QT interval statistics, and are statistics for specified interval values, such as the PP interval. The waveform features may include PR segment statistics and ST segment statistics, and are statistics for specified segment values. The waveform features may include P duration statistics and QRS duration statistics, and are statistics for designated duration values. The waveform features may include P-onset-Peak statistics and P-offset-Peak statistics. The waveform features may include the number of P-waves and the number of R waves.

TABLE 1

| Waveform characteristics | Description |
|---|---|
| P-Peak statistics | Statistics of amplitude value |
| Q-Peak statistics | |
| R-Peak statistics | |
| S-Peak statistics | |
| T-Peak statistics | |
| PP interval statistics | Statistics of interval value |
| QQ interval statistics | |
| RR interval statistics | |
| SS interval statistics | |
| TT interval statistics | |
| PR interval statistics | |
| QT interval statistics | |
| PR segment statistics | Statistics of segment value |
| ST segment statistics | |
| P duration statistics | Statistics of duration value |
| QRS duration statistics | |
| P-Onset-Peak statistics | Statistics of amplitude value |
| P-Offset-Peak statistics | |
| P-wave Number | Number of P-waves |
| R wave Number | Number of R waves |

The ECG preprocessor 100 may extract ECG beat similarity as the ECG feature. The ECG beat similarity is a measure indicating whether a heartbeat is constant. The ECG signal is data measured for a certain period of time (e.g., 10 seconds) and uses a unit called rhythm, and a duration to which the rhythm is divided by each R-peak is called a beat. The ECG beat similarity may be calculated through a beat-wise correlation operation, and may be defined as a mean of a beat-wise correlation coefficient. A method of calculating beat-wise correlation may be various.

Referring to FIG. 4, the ECG preprocessor 100 may extract a first beat (beat 1), which is a reference, from the ECG signal, calculate the similarity between the first beat and the remaining beats (beat 2 to beat N), and extract the mean of the similarities calculated between the beats as the ECG beat similarity. Here, the similarity between the beats may be expressed as the beat-wise correlation coefficient.

The ECG preprocessor 100 may extract fibrillatory wave (F-wave) energy as the ECG feature. In the case of the AF, the F-wave of 4 to 9 Hz is mainly measured, so the energy in the frequency band may be estimated as the F-wave energy. The F-wave energy may be measured variously. The F-wave energy may be calculated, for example, as an F-wave index. The ECG preprocessor 100 may select a beat of interest (BOI) from the ECG signal, extract the F-wave frequency bandwidth (4 to 9 Hz) from the beat of interest, and then calculate the energy of the extracted frequency band. In addition, the ECG preprocessor 100 may calculate the energy of the F-wave frequency band even in certain beats (residual beat of interest, RBOI) excluding the beat of interest, and calculate the ratio of RBOI to BOI as the F-wave index.

The ECG preprocessor 100 may extract the P-wave feature from the ECG signal. By using the P-wave feature, which is highly correlated with the AF, the AF may be accurately predicted from the ECG signal. The ECG preprocessor 100 may extract the P-wave from the ECG signal and use the skewness and kurtosis of the P-wave as the P-wave features.

In this way, the ECG preprocessor 100 may extract various ECG features for each ECG signal and store the ECG feature values obtained from the ECG of the patient, as shown in Table 2. In this case, the ECG preprocessor 100 may store the patient information along with the ECG features in order to consider the individual characteristics in predicting the AF, and the patient information may include, for example, gender and age.

TABLE 2

| Type | Electrocardiogram features | N | Leads | Description |
|---|---|---|---|---|
| Patient | Gender | 1 | — | — |
| information | Age | 1 | — | — |
| Waveform | P-Peak statistics | 4~48 | From 1 to 12 | 4 statistics |
| | Q-Peak statistics | 4~48 | | |
| | R-Peak statistics | 4~48 | | |
| | S-Peak statistics | 4~48 | | |
| | T-Peak statistics | 4~48 | | |
| | PP interval statistics | 4~48 | From 1 to 12 | 4 statistics |
| | QQ interval statistics | 4~48 | | |
| | RR interval statistics | 4~48 | | |
| | SS interval statistics | 4~48 | | |
| | TT interval statistics | 4~48 | | |
| | PR interval statistics | 4~48 | | |
| | QT interval statistics | 4~48 | | |
| | PR segment statistics | 4~48 | From 1 to 12 | 4 statistics |
| | ST segment statistics | 4~48 | | |
| | P duration statistics | 4~48 | From 1 to 12 | 4 statistics |
| | QRS duration statistics | 4~48 | | |
| | P-Onset-Peak statistics | 4~48 | From 1 to 12 | 4 statistics |
| | P-Offset-Peak statistics | 4~48 | | |
| | P-wave Number | 1~12 | From 1 to 12 | |
| | R wave Number | 1~12 | | |
| Electrocardiog | Beat Correlation statistics | 1~12 | From 1 to 12 | Mean of Beat- |
| ram beat | | | | wise |
| Similarity | | | | Correlation |
| | | | | Coefficient |
| Fibrillatory | f-wave index | 1~12 | From 1 to 12 | |
| wave energy | | | | |
| P-wave | P-wave statistics | 2~24 | From 1 to 12 | Skewness, |
| | | | | Kurtosis |

Referring to Table 2, not all 12 ECG signals measured through 12-leads need to be used to predict the AF, and the ECG features may be extracted from signals measured through some leads. Through this, it is possible to predict the AF while significantly reducing the dimension of the data input to the artificial intelligence model 200. For example, the P-QRS-T waveform features, the ECG beat similarity, and the P-wave features may be extracted from signals measured in six leads lead I, lead II, lead V1, lead V3, lead V4, and lead V5, and then, the F-wave energy may be extracted from the measured signals in the four leads I, III, V3, and V5. Of course, the ECG features may be extracted from signals measured in more leads than this, the ECG features may also be extracted from signals measured in other combinations of leads, and the ECG features may be extracted from the signals measured in the Lead I and the Lead II.

The feature values of each variable may be calculated by four statistics functions (minimum value, maximum value, mean, and standard deviation), and the type or number of statistics functions may change. For example, the P-Peak statistics, one of the waveform features, is extracted from the ECG signals measured in 6-leads. Since an amplitude value of the P-peak is calculated using four statistics functions, a total of 24 may be expressed as the feature values. Since the ECG beat similarity is extracted for each ECG signal measured in the 6-leads, it may be expressed as a total of 6 feature values. Since the F-wave energy is extracted for each ECG signal measured in the 4-leads, it may be expressed as a total of 4 feature values. Since the P-wave feature is the skewness and kurtosis of each ECG signal measured in the 6-leads, it may be expressed as a total of 12 feature values.

Here, all the ECG features obtained for one ECG do not need to be used to generate the input data of the artificial intelligence model 200, and the input data of the artificial intelligence model 200 may be defined in various ways by a combination of the ECG features. Here, according to the analysis of variance (ANOVA) and F-test results for the serial ECG model 210, the difference in P-wave amplitude between the two NSR ECGs is analyzed as the most powerful ECG parameter that distinguishes the AF and the NSR. Therefore, it is possible to improve the prediction performance of the AF through the input data including the P-wave amplitude difference (difference in P-peak statistics).

For example, the input data of the artificial intelligence model 200 may be composed of gender, age, and at least some P-QRS-T waveform features, and at least some of the P-QRS-T waveform features may include peak statistics of each of P, Q, R, S, and T, interval statistics of each of P, Q, R, S, and T, PR interval statistics, and QRS duration statistics. In addition to gender, age, and at least some of the P-QRS-T waveform features, the remaining P-QRS-T waveform features, the ECG beat similarity, the F-wave energy, and the P-wave features may be optionally included in the input data of the artificial intelligence model 200.

The input data of the serial ECG model 210 or the single ECG model 220 is composed of at least some of the ECG features, and input data values may be filled differently. That is, the feature difference between the ECG pairs measured at a certain period of time may be input to the serial ECG model 210 that receives the changes in ECGs. The features of the single ECG may be input to the single ECG model 220 as they are.

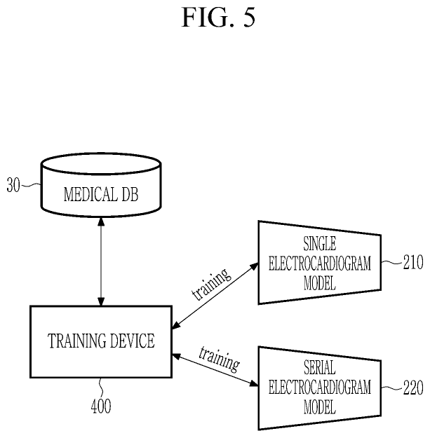

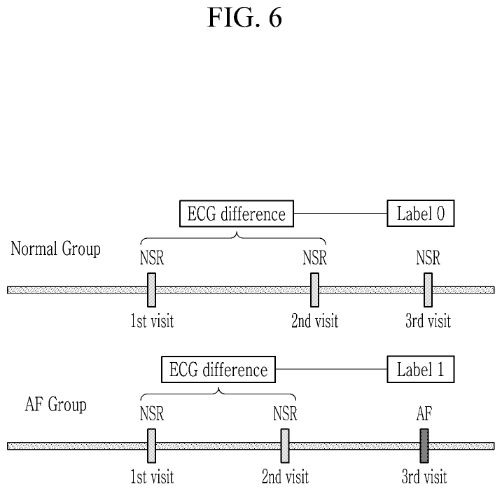

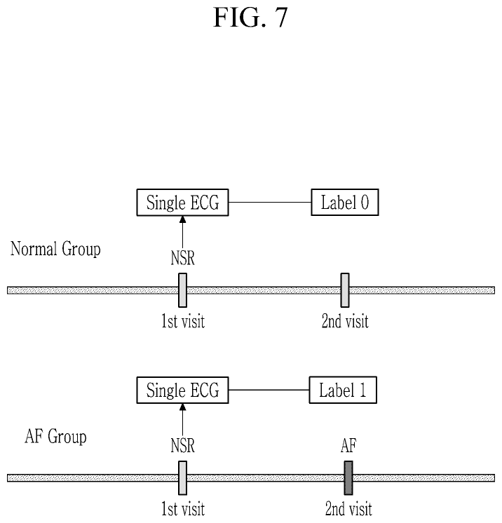

FIG. 5 is a diagram describing training of the artificial intelligence model according to an embodiment. FIG. 6 is a diagram illustrating a method of generating training data for a serial ECG model according to an embodiment. FIG. 7 is a diagram describing a method of generating training data for a single ECG model according to an embodiment.

Referring to FIG. 5, a training device 400 may generate training data for training the artificial intelligence model 200 and train the artificial intelligence model 200. The artificial intelligence model 200 may include the serial ECG model 210 and may further include the single ECG model 220.

The training device 400 extracts the ECGs of the patients from the medical DB 30. The training device 400 may classify patients who have at least one NSR ECG prior to AF ECG and for whom the AF diagnosis has been confirmed based on the medical records or diagnosis codes into an AF group. The training device 400 may classify patients with at least two NSR ECGs and no clinical history of AF diagnosis into an NSR group. The training device 400 may consider one of the two ECGs as an invalid ECG when the ECG measurement period of time (e.g., 1 month) is shorter than the minimum period (e.g., 1 month) or longer than the maximum period of time (e.g., 1 year), and classify patients.

Referring to FIG. 6, for the training of the serial ECG model 210, the training device 400 may extract patients with three or more valid ECGs from the NSR group and the AF group. The training device 400 may extract ECGs of patients whose ECGs measured at the first visit, the second visit, and the third visit are all NSR, generate a difference between the first ECG and the second ECG as the input data, and may tag the label 0 to the input data. In addition, the training device 400 may extract the ECGs of the patients from the AF group whose two ECGs measured before the AF ECG are NSR, generate the difference between the first NSR ECG and the second NSR ECG as the input data, and tag the label 1 to the input data. The training device 400 may use the input data tagged with the label 1 and label 0, so the serial ECG model 210 may train the relationship between the input data and the tagged label, and may be trained to output a predicted value between 0 and 1 for the input. Through this, the serial ECG model 210 may receive the ECG difference and output the probability of onset-AF. The serial ECG model 210 may be trained to predict the probability of onset-AF for each period (e.g., 3 months, 6 months, 12 months, etc.), and may be composed of a plurality of sub-models as needed.

Referring to FIG. 7, for the training of the single ECG model 220, the training device 400 may extract patients with two or more valid ECGs from the NSR group and the AF group. The training device 400 may extract first NSR ECG of patients whose ECGs measured at the first visit and the second visit are all NSR, generate the features of the first ECG as the input data, and may tag the label 0 to the input data. In addition, the training device 400 may extract, from the AF group, the ECG of the patient whose ECG measured before the AF ECG is NSR, generate the NSR ECG features as the input data, and tag the label 1 to the input data. The training device 400 may use the input data tagged with the label 1 and label 0, so the single ECG model 220 may train the relationship between the input data and the tagged label, and may be trained to output a predicted value between 0 and 1 for the input. Through this, the single ECG model 220 may predict the probability that the AF will occur next from the single ECG. The single ECG model 220 may be trained to predict the probability of onset-AF for each period (e.g., 3 months, 6 months, 12 months, etc.), and may be composed of a plurality of sub-models as needed.

The training data may be divided into a training set, a validation set, and a test set. In this case, the training set, the validation set, and the test set may be classified so that the same patients are not mixed.

Meanwhile, due to the insufficient number of patients with NSR ECG before the AF ECG, the training data for the label 1 is actually bound to be significantly smaller than the training data for the label 0. In order to improve the performance of the artificial intelligence model, this data imbalance problem should be resolved. In general, the data imbalance problem is solved through data augmentation, but even if the NSR ECG of the AF patient is synthesized and generated, it is difficult to reflect the subtle changes in ECGs that cause the AF. Therefore, there is a limitation that it is difficult for the artificial intelligence model trained with augmented data to accurately predict the onset-AF possibility. Therefore, the data imbalance problem may be resolved by setting a learning rate of the artificial intelligence model.

Meanwhile, a blanking period and optimal follow up period may be found through the artificial intelligence model 200. The blanking period may be defined as the minimum period during which the artificial intelligence model may distinguish the subtle changes in the atrium while atrial remodeling occurs.

In the case of the single ECG model 220, the artificial intelligence model is trained with data excluding the NSR ECG for a certain period of time (1 month, 2 months, 3 months, etc.) from the AF ECG, and the performance of the artificial intelligence model may be compared to determine the optimal blanking period. As a result of the experiment, the blanking period may be defined as 1 month, 2 months or more. There is a high possibility of detecting subtle changes due to the atrial remodeling before the onset of atrial fibrillation.

For the serial ECG model 210, one NSR ECG may be selected to be included in the defined blanking period, and the other NSR ECG may be selected to be an ECG before the blanking period to train the artificial intelligence model and compare the performance of the artificial intelligence model. As a result of the experiment, AUROC of the serial ECG model 210 was 0.917 over a follow up period within 1 month, 0.936 over a 1-month follow up period, 0.944 over a 3-month follow up period, and 0.935 over a 12-month follow up period, so the model performance for predicting new onset AF shows the highest predictive value over the 3-month follow up period. Through this, the optimal follow up period between the ECGs may be defined as 3 months or longer to reflect the atrial remodeling.

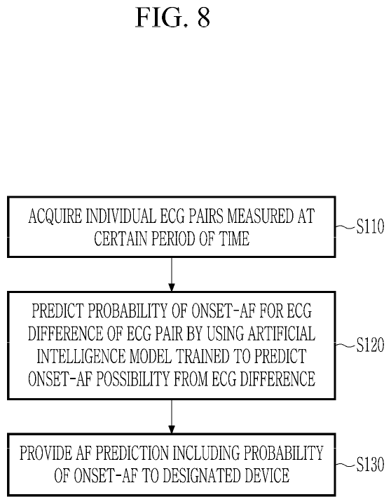

FIG. 8 is a flowchart illustrating an operating method of an apparatus for predicting AF according to an embodiment.

Referring to FIG. 8, the apparatus 10 for predicting AF acquires individual ECG pairs measured at a certain period of time (S110). For example, one of the ECG pairs may be an ECG measured at this visit, and the other may be an ECG measured at a previous visit. When there are multiple previous visits, the apparatus 10 for predicting AF may use the ECG measured before a specified period (e.g., 3 months) from this visit (baseline time) to predict the AF. Even if there is the ECG measured at the previous visit, when if it is an ECG measured before a specified period (e.g., 1 year) from this visit (baseline time), the apparatus 10 for predicting AF may not use the ECG as one of the ECG pairs.

The apparatus 10 for predicting AF predicts the probability of onset-AF for the ECG difference of the ECG pair by using the artificial intelligence model trained to predict an onset-AF possibility from the ECG difference (S120). The artificial intelligence model may be the serial ECG model 210. The apparatus 10 for predicting AF may remove noise or remove outliers from each ECG included in the ECG pair according to a designated procedure, extract the ECG features from the ECG signals, and generate the input data for the artificial intelligence model. The serial ECG model 210 may use a feature difference between the ECG pair expressed numerically as the input data. The ECG features may be composed of at least some P-QRS-T waveform features, and may include peak statistics of each of P, Q, R, S, and T, interval statistics of each of P, Q, R, S, and T, PR interval statistics, and QRS duration statistics. In addition, the ECG features may further include at least some of the remaining P-QRS-T waveform features, the ECG beat similarity, the F-wave energy, and P-wave features. Meanwhile, the input data of the serial ECG model 210 may further include individual information (gender, age) along with the difference in feature values of the ECG pairs.

The apparatus 10 for predicting AF provides the AF prediction including the probability of onset-AF to the designated device (S130). The probability of onset-AF may be predicted for at least one period. The probability of onset-AF is a way of expressing the possibility of onset-AF, and may also be expressed as a score, a risk level (high, middle, low), etc. The AF prediction may further include performance indicators (e.g., sensitivity, specificity, accuracy, etc.) for predicted values. The probability of onset-AF may be predicted for at least one period. The apparatus 10 for predicting AF may further provide the decision-making assistance information related to the AF prediction. The decision-making assistance information may include recommended follow up clinical procedures based on the AF prediction. The designated device may be, for example, the terminal 40 used by medical personnel, but is not limited thereto.

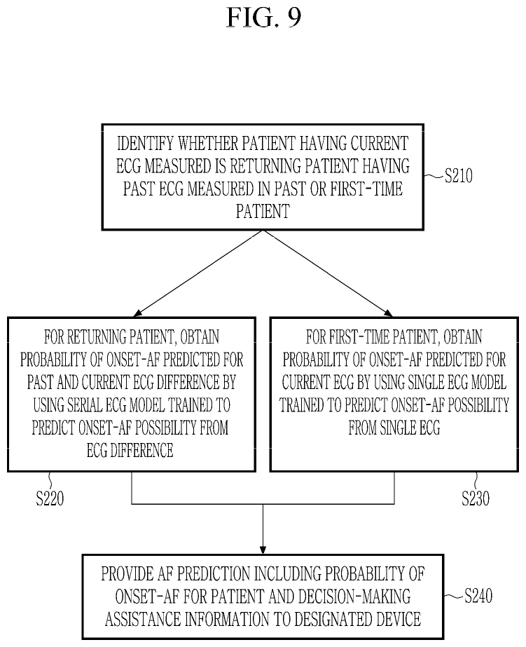

FIG. 9 is a flowchart illustrating an operating method of an apparatus for predicting AF according to another embodiment.

Referring to FIG. 9, the apparatus 10 for predicting AF identifies whether a patient having a current ECG measured is a returning patient having a past ECG measured in the past or a first-time patient (S210). The apparatus 10 for predicting AF may refer to the medical DB 30 and determine a patient with a valid ECG measured at a previous visit as a returning patient. The valid ECG may be determined depending on the time when the ECG is measured.

The apparatus 10 for predicting AF, for the returning patient, obtains the probability of onset-AF predicted for the past and current ECG difference by using the serial ECG model 210 trained to predict the onset-AF possibility from the ECG difference. (S220).

The apparatus 10 for predicting AF, for the first-time patient, obtains the probability of onset-AF predicted for the current ECG by using the single ECG model 220 trained to predict the onset-AF possibility from the single ECG (S230).

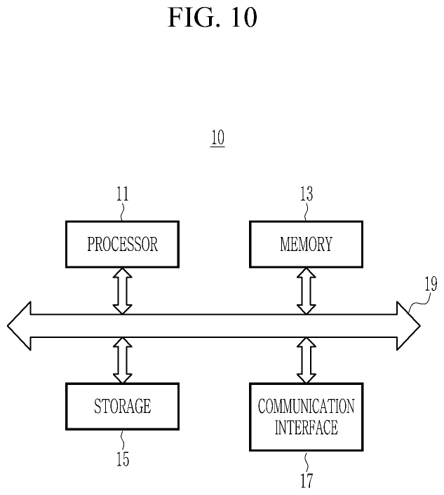

The apparatus 10 for predicting AF provides the AF prediction including the probability of onset-AF for the patient and decision-making assistance information to the designated device (S240). The apparatus 10 for predicting AF may determine the decision-making assistance information based on the probability of onset-AF and the identified patient type. For example, the apparatus 10 for predicting AF may provide recommended follow up clinical procedures (e.g., active ECG monitoring test, follow up termination without additional testing, re-performing ECG test after a certain period of time, etc.) according to the probability of onset-AF as the decision-making assistance information. It is assumed that the recommended follow up clinical procedures according to the probability of onset-AF are predetermined according to the clinical protocol. The apparatus 10 for predicting AF may recommend the active ECG monitoring test when the probability of onset-AF is higher than the reference. The apparatus 10 for predicting AF may recommend a second ECG test 3 months later when the patient is the first-time patient and the probability of onset-AF is below the reference. The apparatus 10 for predicting AF may recommend the ECG test every 6 months if the patient is the returning patient who received the ECG test within 3 months after the first visit and the probability of onset-AF is below the reference. The designated device may be, for example, the terminal 40 used by medical personnel, but is not limited thereto. FIG. 10 is a hardware configuration diagram of the apparatus for predicting AF according to an embodiment.

Referring to FIG. 10, the apparatus 10 for predicting AF may be implemented as a computing device operated by at least one processor. The apparatus 10 for predicting AF includes one or more processors 11, a memory 13 for loading a computer program executed by the processor 11, a storage 15 for storing computer programs and various data, a communication interface 17, and a bus 19 connecting them. In addition, the apparatus 10 for predicting AF may further include various components.

The processor 11 is a device that controls the operation of the apparatus 10 for predicting AF, and may be various types of processors that process instructions included in a computer program. The processor 11 may be configured to include, for example, a central processing unit (CPU), a micro processor unit (MPU), a micro controller unit (MCU), a graphic processing unit (GPU), or at least one of any type of processors well known in the art of the present disclosure.

The memory 13 stores various data, instructions and/or information. The memory 13 may load the corresponding computer program from the storage 15 so that the instructions described to execute the operations of the present disclosure are processed by the processor 11. The memory 13 may be, for example, read only memory (ROM), random access memory (RAM), etc.

The storage 15 may non-temporarily store computer programs and various data. The storage 15 may be configured to include a nonvolatile memory, such as a read only memory (ROM), an erasable programmable ROM (EPROM), an electrically erasable programmable ROM (EEPROM), and a flash memory, a hard disk, a removable disk, or any well-known computer-readable recording medium in the art to which the present disclosure belongs.

The communication interface 17 may be a wired/wireless communication module that supports wired/wireless communication.

The bus 19 provides communication between components of the apparatus 10 for predicting AF.

The computer program includes instructions executed by the processor 11 and stored on a non-transitory computer readable storage medium. The instructions cause processor 11 to execute the present disclosure operation. The computer program may be downloaded through the network or sold in the product form. The artificial intelligence model 200 may be implemented as the computer program executed by the processor 11.

The computer program may include instructions executed by the processor 11 to cause the apparatus 10 for predicting AF to: acquire individual ECG pairs measured at a certain period of time; obtaining a probability of onset-AF for an ECG difference between the ECG pairs using an artificial intelligence model trained to predict an onset-AF possibility from the ECG difference; provide the AF prediction including the probability of onset-AF to the designated device. In addition, the computer program may include instructions executed by the processor 11 to cause the apparatus 10 for predicting AF to: identify whether the patient is the returning patient who has measured the ECG in the past or is the first-time patient; obtaining the probability of onset-AF using the serial ECG model 210 when the patient is the returning patient, and obtaining the probability of onset-AF using the single ECG model 220 when the patient is the first-time patient; provide AF prediction including the probability of onset-AF and the decision-making assistance information to the designated device.

In this way, according to the present disclosure, it is possible to accurately select patients being new onset-AF in the future through the evolved artificial intelligence model considering individual characteristics.

According to the present disclosure, it is possible to support medical personnel's decision-making such as active electrocardiogram monitoring or preemptive anticoagulation treatment for patients with a high probability of onset-AF, and increase the AF diagnosis rate.

According to the present disclosure, it is possible to recommend the appropriate follow up clinical procedures depending on the possibility of onset-AF and determine the optimal follow up period (e.g. 3 months) considering the accuracy of AF prediction.

The embodiment of the present disclosure described above is not implemented only through the method and apparatus, and may be implemented through a program for realizing a function corresponding to the configuration of the embodiment of the present disclosure or a recording medium in which the program is recorded.

Although embodiments of the present disclosure have been described in detail hereinabove, the scope of the present disclosure is not limited thereto, but may include several modifications and alterations made by those skilled in the art using a basic concept of the present disclosure as defined in the claims.

What is claimed is:

1. An apparatus for predicting atrial fibrillation (AF) operated by at least one processor, comprising:
an electrocardiogram (ECG) preprocessor configured to acquire individual ECG pairs measured at a certain period of time and generate a difference between the ECG pairs as input data for an artificial intelligence model;
an artificial intelligence model configured to be trained to predict an onset-AF possibility from an ECG difference and output a probability of onset-AF predicted from the input data; and
a prediction information provider configured to provide AF prediction including the probability of onset-AF to a designated device.

2. The apparatus of claim 1, wherein the ECG preprocessor is configured to extract ECG features from ECG signals of each ECG included in the ECG pair, and generate a feature difference between the two ECGs as the input data.

3. The apparatus of claim 2, wherein the ECG features include P-QRS-T waveform features.

4. The apparatus of claim 3, wherein the ECG features further include at least one of ECG beat similarity, fibrillation wave energy, and P-wave features.

5. The apparatus of claim 1, wherein the input data further includes at least one of an individual gender and age.

6. The apparatus of claim 1, wherein the artificial intelligence model is trained to learn a relationship between an input and a label tagged in the input using training data to output a value between 0 and 1 predicted for the input data, and
the training data is generated using ECGs of patients whose first ECG and second ECG measured at a first visit and a second visit are normal, and whose third ECG measured at a third visit is normal or has AF, the input is a difference between the first ECG and the second ECG, and the label is given according to the third ECG of the patient.

7. The apparatus of claim 1, wherein the AF prediction further includes a performance indicator for the probability of onset-AF.

8. The apparatus of claim 1, wherein the prediction information provider is configured to provide decision-making assistance information related to the AF prediction.

9. An operating method of an apparatus for predicting atrial fibrillation (AF) operated by at least one processor, comprising:
acquiring individual electrocardiogram (ECG) pair measured at a certain period of time, and
predicting a probability of onset-AF for an ECG difference between the ECG pairs using an artificial intelligence model trained to predict an onset-AF possibility from the ECG difference.

10. The operating method of claim 9, wherein the artificial intelligence model is trained to learn a relationship between an input and a label tagged in the input using training data to output a value between 0 and 1 predicted for input data, and
the training data is generated using ECGs of patients whose first ECG and second ECG measured at a first visit and a second visit are normal, and whose third ECG measured at a third visit is normal or has the AF, the input is a difference between the first ECG and the second ECG, and the label is given according to the third ECG of the patient.

11. The operating method of claim 9, further comprising:
extracting ECG features from ECG signals of each ECG included in the ECG pair, and generating a feature difference between the two ECGs as input data of the artificial intelligence model.

12. The operating method of claim 11, wherein the ECG features include P-QRS-T waveform features.

13. The operating method of claim 12, wherein the ECG features further include at least one of ECG beat similarity, fibrillation wave energy, and P-wave features.

14. The operating method of claim 11, wherein the input data further includes at least one of an individual gender and age.

15. The operating method of claim 9, further comprising:
predicting the AF including the probability of onset-AF; and
providing decision-making assistance information related to the AF prediction to a designated device.

16. The operating method of claim 15, wherein the AF prediction further includes a performance indicator for the probability of onset-AF.

17. An operating method of an apparatus for predicting atrial fibrillation (AF) operated by at least one processor, comprising:
identifying whether a patient having a current ECG measured is a returning patient having a past ECG measured in the past or a first-time patient;
for the returning patient, obtaining a probability of onset-AF predicted for a past and current ECG difference of the patient using a serial ECG model trained to predict an onset-AF possibility from the ECG difference;
for the first-time patient, obtaining a probability of onset-AF predicted for the current ECG of the patient using a single ECG model trained to predict an onset-AF possibility from the single ECG; and
providing an AF prediction including the probability of onset-AF for the patient to a designated device.

18. The operating method of claim 17, wherein the serial ECG model is trained to learn a relationship between an input and a label tagged in the input using training data to output a value between 0 and 1 predicted for the ECG difference of the patient, and
the training data is generated using ECGs of patients whose first ECG and second ECG measured at a first visit and a second visit are normal and whose third ECG measured at a third visit is normal or has the AF, the input is a difference between the first ECG and the second ECG, and the label is given according to the third ECG of the patient.

19. The operating method of claim 17, wherein the single ECG model is trained to learn a relationship between an input and a label tagged in the input using training data to output a value between 0 and 1 predicted for the input data, and
the training data is generated using ECGs of patients whose first ECG measured at a first visit is normal, and whose second ECG measured at a second visit is normal or has the AF, the input is features of the first ECG, and the label is given according to the second ECG of the patient.

20. The operating method of claim 17, further comprising:
providing decision-making assistance information related to the AF prediction to the designated device.
